(19) United States
(12) Patent Application Publication
(10) Pub. No.: US 2012/0070475 A1
(43) Pub. Date: Mar. 22, 2012

(54) Zero Calorie Polyphenol Aqueous Dispersions

(75) Inventors: Naijie Zhang, Ridgefield, CT (US); William Mutilangi, Peekskill, NY (US)

(73) Assignee: PEPSICO, INC, Purchase, NY (US)

(21) Appl. No.: 13/307,405

(22) Filed: Nov. 30, 2011

Related U.S. Application Data
(63) Continuation-in-part of application No. 13/036,161, filed on Feb. 28, 2011.
(60) Provisional application No. 61/312,694, filed on Mar. 11, 2010.

(51) Int. Cl. A61K 31/353; A61P 17/18; A61K 31/12; A61K 9/14; A61K 31/05; A61K 31/7048

(52) U.S. Cl. 424/400; 514/456; 514/733; 514/27; 514/679

(57) ABSTRACT

A method for stably dispersing microparticulated water insoluble bioactive polyphenol in a beverage by combining the bioactive polyphenol dissolved in an alkaline solution with an aqueous solution of at least one dispersion stabilizer. A composition comprising dispersed microparticulated water insoluble bioactive polyphenol(s) and a dispersion stabilizer.

This application is continuation-in-part of application Ser. No. 13/036161 filed Feb. 28, 2011, which claims priority to application Ser. No. 61/312,694 filed Mar. 11, 2010, the disclosures of which are incorporated by reference in their entirety.

### FIELD OF THE INVENTION

The present invention relates generally to a method for dispersing water insoluble polyphenol particles in a liquid medium such as a beverage, and a composition containing polyphenol particles and dispersion stabilizer(s).

### BACKGROUND

Numerous clinical studies have linked polyphenols with reduced cardiovascular disease, cancer, osteoporosis, and with other health benefits such as their antioxidant, anti-inflammatory, antibacterial, antiviral, and anti-allergenic properties. Quercetin, in particular, is considered a powerful antioxidant. A number of studies showed that quercetin is effective for the prevention of various diseases.

Most polyphenols are highly rigid and crystalline in structure and are consequently water insoluble. Polyphenols may be extracted from plants. For example, quercetin is a natural, plant-derived, polyphenol. In particular, quercetin is the aglycone form of a number of other flavonoid glycosides (polyphenols), such as rutin and quercitrin, found in citrus fruit, cranberries, blueberries, buckwheat, onions, and other vegetables, fruits, and green plants. The chemical structure of quercetin is illustrated below:

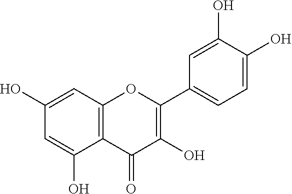

### 2-(3,4-dihydroxyphenyl)-3,5,7-trihydroxy-4H-chromen-4-one

It is desired to use polyphenols as a nutritional supplement in food products such as beverages. Often such polyphenols are difficult to disperse and mix into beverage products at efficacious concentrations. They will simply settle to the bottom of the container holding the beverage. For example, quercetin is typically obtained as a powder and is insoluble in water. When added to liquid media, quercetin usually agglomerizes and settles to the bottom of the beverage, thereby resulting in a product that is not visually appealing to the consumer.

Therefore, a need exists in the food and beverage industry to provide the consumer with a food product containing bioactive polyphenols, wherein the polyphenols are dispersed and remain stably suspended in an aqueous solution.

### BRIEF SUMMARY

The present invention relates to a method for dispersing polyphenol particles in a beverage to create a stable dispersion and to a composition for a beverage containing a stable dispersion of polyphenol particles.

In co-pending application (Ser. No. 13/036161, filed Feb. 28, 2011), a polyphenol such as quercetin is dissolved in hot glycerol or propylene glycol followed by addition into an aqueous solution containing a stabilizer such as gellan gum resulting in stable fine dispersion. In addition to the cost and operational challenges related to high temperature processing, the use of polyols limits application of this technology in the development of zero calorie beverages since polyols contribute 4-5 calories per gram. The present invention is simpler and enables delivery of water insoluble polyphenol particles without use of caloric organic solvents and at relatively lower temperature.

Co-pending application Ser. No. 13/036161 also describes dissolving a polyphenol in an alkaline solution. It was recently discovered that the process temperature plays a vital role in maintaining polyphenol structure in alkaline solution and surprisingly, the lower the temperature, the better the chemical structure stability. In addition to quercetin, the present method also delivers stable beverage dispersions of other polyphenols such as curcumin, rutin, resveratrol, naringenin, and hesperedin. Structural integrity of the polyphenol dispersions prepared by this invention has been confirmed by FT-IR, proton NMR, carbon-13 NMR and mass spectrometry.

In particular, a) microparticulated water insoluble bioactive polyphenol particles are dissolved in an alkaline solution at a temperature of below 30° C., and b) the dissolved alkaline bioactive polyphenol solution is then added to aqueous solution containing an effective amount of at least one dispersion stabilizer to create a stable dispersion of the water insoluble bioactive polyphenol particles, wherein the polyphenol particles are microparticulated.

### DETAILED DESCRIPTION OF THE INVENTION

The present invention relates to a method for dispersing microparticulated water insoluble bioactive polyphenol particles in a liquid medium, such as a beverage, by stabilizing the particles with at least one dispersion stabilizer. Beverages, prepared with the water insoluble bioactive polyphenol particles with a dispersion stabilizer, contain fine, stably dispersed (suspended) particles.

Suitable polyphenols include quercetin, eriocitrin, neoeriocitrin, narirutin, naringin, hesperidin, hesperetin, neohesperidin, neoponcirin, poncirin, rutin, isorhoifolin, rhoifolin, diosmin, neodiosmin, sinensetin, nobiletin, tangeritin, catechin, catechin gallate, epigallocatechin, epigallocatechin gallate, anthocyanin, heptamethoxyflavone, curcumin, resveratrol, naringenin, tetramethoxyflavone, kaempferol, and rhoifolin. Other suitable polyphenols include oolong tea polymerized polyphenols.

Suitable polyphenols also include flavonoids including flavonones, flavones, dihydroflavonols, flavonols, flavandiols, leucoanthocyanidins, isoflavonoids, and neoflavonoids. See for example, Naturally Occurring Bioactive Compounds Edited by Mahendra Rai, Maria Cecillia Carpinella, 2006. Bioactive Compounds in Foods Edited by John Gilbert and Hamide Z. Senyuva. Bioactive Compounds From Plants, Volume 154, John Wiley and Sons, 1990.

It was discovered that the temperature significantly affected the chemical stability of polyphenol particles in alkaline solutions. Polyphenol particles in an alkaline solution have a higher stability in the alkaline solution if the alkaline solution is maintained at lower temperatures (e.g. 7° C.) in contrast to temperatures at room temperature (e.g. 25° C.) or above where polyphenols such as quercetins will decompose even if stored for only 45 minutes.

It was further discovered that the type of pH buffer affected stability of the polyphenols dissolved in an alkaline solution. For example, an alkaline polyphenol solution at pH 10 is more stable with the combination of dipotassium phosphate and sodium hydroxide than with sodium hydroxide solution alone.

It was further discovered that, during the addition of an alkaline polyphenol solution into an aqueous solution of dispersion stabilizer, the pH is critical to avoid polyphenol structure change and color change. When an alkaline polyphenol solution is added into an aqueous solution of dispersion stabilizer under high shear mixing, the pH of the resulting dispersion should be maintained between 3 and 5, or 3.5 to 4, or about 4. The temperature of the dispersion stabilizer solution should be kept below 50° C. under high shear mixing.

Further, the dispersion stabilizers, such as gellan gum, should be hydrated prior to adding the alkaline polyphenol solution. For example, gellan gum may be hydrated by mixing gellan gum with water at a temperature below 50° C. until fully hydrated, for example 25° C. for 20 minutes. After the dispersion stabilizer is hydrated, it is subjected to high shear mixing.

The method does not require organic solvents such as polyols, nor are there any organic solvents such as polyols included in the method of making the dispersion and resulting compositions.

In one aspect of the invention, a polyphenol is dissolved in an alkaline solution. The temperature of the alkaline solution is less than 30° C., generally 2 to 20° C., 5 to 15 ° C., or 5 to 10° C. The pH of the resulting solution is from 9 to 12, or 9.5 to 11.5, or 10 to 11.

Suitable alkaline solutions include water and sodium hydroxide, potassium hydroxide, ammonium hydroxide, sodium carbonate, sodium hydrogenate carbonate, sodium bicarbonate, sodium hydrogen orthophosphate, potassium chloride, citric acid, or mixtures thereof. In some aspects a combination of sodium hydroxide and potassium hydroxide is used. The amount of alkaline compound is used in an amount sufficient to obtain the desired pH level. Other suitable combinations include, but are not limited to, sodium carbonate and sodium hydrogen carbonate; sodium bicarbonate and sodium hydroxide; sodium hydrogen orthophosphate and sodium hydroxide; potassium chloride and sodium hydroxide; and citric acid and sodium hydroxide.

When completely dissolved, the polyphenol in alkaline solution is added into an aqueous solution containing a dispersion stabilizer with high shear mixing or agitation. The aqueous dispersion has a pH of 3 to 5 and a temperature below 50° C. and the pH of the aqueous dispersion is maintained at 3 to 5 and a temperature of below 50° C. during addition of the alkaline solution. The resulting polyphenol comes out of solution resulting in fine particle dispersion.

The high shear mixing or agitation may be achieved by any suitable means such as a IKA T25 mixer or SILVERSON L4RT-A. The stabilizer solution should have a pH between 3.5 and 5, or 3.8 to 4.5, or about 4.

The at least one dispersion stabilizer can be a biopolymer or a modified polysaccharide such as gellan gum, pectin, carrageenan, ghatti gum, acacia gum, guar gum, xanthan gum, locust gum, agar, starch, alginate, cellulose, protein, hydrolyzed protein, modified starch, carboxylmethyl cellulose (CMC) or the combination thereof.

Preferably the biopolymers are charged polymers such as carboxyl-containing polymers and sulfate-containing polymers. It was discovered that anionic or cationic biopolymers such as pectin, gellan gum, carrageenan, gum arabic, ghatti gum, CMC, whey protein isolate showed better dispersion stability than non-ionic polymers. It is believed that quercetin absorbed on a charged polymer exhibits stable aqueous dispersion due to electrostatic, steric repulsion between the particles. There is no settling/aggregation after stored at ambient conditions. The inventors discovered that the length of time that the quercetin stays dispersed in the liquid media varies depending on the type(s) of dispersion stabilizer(s) used. For example, the quercetin can stay dispersed in a beverage for about 12 hours to about six months or longer, depending on the dispersion stabilizer(s) used.

The stable dispersion contains from 0.1 to 10 weight % polyphenol. Subsequently, water is added to dilute the concentrated dispersion to yield a stable dispersion having a concentration between 0.001-5.0 wt. %. The dispersion stabilizer is present in an amount sufficient to disperse (suspend) the microparticulated polyphenol in the beverage.

The polyphenol comes out of solution as is microparticulates. “Microparticulated” or “microparticulate” as used in the instant application means a small particle ranging in size from about 0.1 microns to about 50 microns with an average particle size below about 10 microns, in particular less than 3 microns or less than 1 micron. For example, at least 90% of the particles have a particle size less than 50 microns and 80% of the particles have a particle size less than 3 microns.

The pH of the diluted dispersion may then be adjusted to less than 7, typically 2.5 to 6, depending on the type of beverage. For example the stabilized solution may be acidified using a food grade acid to a pH of from 2.5 to 5, in particular to a pH of from 3 to 4. Suitable food acids include, but are not limited to, citric acid and phosphoric acid.

Food-grade preservatives such as, but not limited to, sodium benzoate, potassium sorbate, or lauric arginate, may be added.

The polyphenol comprises 0.01 to 1 wt % of the final beverage composition, for instance 0.01 to 0.5 wt %, or 0.01 to 0.2 wt %.

The beverage may be any suitable beverage including, but not limited to, juices, carbonated soft drinks, water, dairy, and isotonic beverages. One of ordinary skill in the art of the chemical and food sciences would recognize that any polyphenol may be used in accordance with the present invention.

Although the beverage appears relatively viscous and/or thick upon adding the polyphenol to the beverage, by using the solubilized/dispersed polyphenol in conjunction with the dispersion stabilizer, as disclosed herein, the beverage had a thin consistency upon consumption. This unexpected result, the thin consistency notwithstanding the viscous visual appearance, is also advantageous, as it leads to consumer likability and acceptance of the beverage.

The method of the present invention may also include one or more additional ingredients selected from the group consisting of carbohydrates, salts, salt blends, flavors, colors, Vitamin B3, Vitamin C, non-nutritive and/or nutritive sweeteners and combinations of these ingredients.

Sweeteners of beverage embodiments of the invention include caloric carbohydrate sweeteners, natural high-potency sweeteners, synthetic high-potency sweeteners, other sweeteners, and combinations thereof. With the guidance provided herein, a suitable sweetening system (whether a single compound or combination thereof) can be selected.

Examples of suitable caloric carbohydrate sweeteners include sucrose, fructose, glucose, erythritol, maltitol, lactitol, sorbitol, mannitol, xylitol, D-tagatose, trehalose, galactose, rhamnose, cyclodextrin (e.g., α-cyclodextrin, β-cyclodextrin, and γ-cyclodextrin), ribulose, threose, arabinose, xylose, lyxose, allose, altrose, mannose, idose, lactose, maltose, invert sugar, isotrehalose, neotrehalose, palatinose or isomaltulose, erythrose, deoxyribose, gulose, idose, talose, erythrulose, xylulose, psicose, turanose, cellobiose, glucosamine, mannosamine, fucose, glucuronic acid, gluconic acid, glucono-lactone, abequose, galactosamine, xylo-oligosaccharides (xylotriose, xylobiose and the like), gentio-oligoscaccharides (gentiobiose, gentiotriose, gentiotetraose and the like), galacto-oligosaccharides, sorbose, nigero-oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), lactulose, melibiose, raffinose, rhamnose, ribose, isomerized liquid sugars such as high fructose corn/starch syrup (e.g., HFCS55, HFCS42, or HFCS90), coupling sugars, soybean oligosaccharides, and glucose syrup.

Other sweeteners suitable for use in embodiments provided herein include natural, synthetic, and other high-potency sweeteners. As used herein, the phrases “natural high-potency sweetener,” “NHPS,” “NHPS composition,” and “natural high-potency sweetener composition” are synonymous. “NHPS” means any sweetener found in nature which may be in raw, extracted, purified, treated enzymatically, or any other form, singularly or in combination thereof and characteristically has a sweetness potency greater than sucrose, fructose, or glucose, yet has fewer calories. Non-limiting examples of NHPS's suitable for embodiments of this invention include rebaudioside A, rebaudioside B, rebaudioside C (dulcoside B), rebaudioside D, rebaudioside E, rebaudioside F, dulcoside A, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobtain, baiyunoside, osladin, polypodoside A, pterocaryoside A, pterocaryoside B, mukurozioside, phlomisoside I, periandrin I, abrusoside A, and cyclocarioside I.

NHPS also includes modified NHPS's. Modified NHPS's include NHPS's which have been altered naturally. For example, a modified NHPS includes, but is not limited to, NHPS's which have been fermented, contacted with enzyme, or derivatized or substituted on the NHPS. In one embodiment, at least one modified NHPS may be used in combination with at least one NHPS. In another embodiment, at least one modified NHPS may be used without a NHPS. Thus, modified NHPS's may be substituted for a NHPS or may be used in combination with NHPS's for any of the embodiments described herein. For the sake of brevity, however, in the description of embodiments of this invention, a modified NHPS is not expressly described as an alternative to an unmodified NHPS, but it should be understood that modified NHPS's can be substituted for NHPS's in any embodiment disclosed herein.

As used herein, the phrase “synthetic sweetener” refers to any composition that is not found in nature and is a high potency sweetener. Non-limiting examples of synthetic sweeteners suitable for embodiments of this invention include sucralose, acesulfame potassium (acesulfame K or aceK) or other salts, aspartame, alitame, saccharin, neohesperidin dihydrochalcone, cyclamate, neotame, N-[3-(3-hydroxy-4-methoxyphenyl)propyl]-L-α-aspartyl]-L-phenylalanine 1-methyl ester, N-[3-(3-hydroxy-4-methoxyphenyl)-3-methylbutyl]-L-α-aspartyl]-L-phenylalanine 1-methyl ester, N-[3-(3-methoxy-4-hydroxyphenyl)propyl]-L-α-aspartyl]-L-phenylalanine 1-methyl ester, and salts thereof.

The method described herein is advantageous as it successfully suspends water-insoluble bioactive polyphenol particles in the beverage and thereby deters settling of the water-insoluble bioactive polyphenol particles to the bottom of the beverage's packaging.

The present invention also relates to compositions comprising dispersed water-insoluble bioactive polyphenol particles and at least one dispersion stabilizer, wherein the particles are microparticulated. In one aspect of this invention, about 90% of the water-insoluble bioactive polyphenol particles is below about 50 microns and the at least one dispersion stabilizer is present in an amount sufficient to suspend the water-insoluble bioactive polyphenol particles in a liquid medium.

The following examples are specific embodiments of the present invention, but are not intended to limit the invention.

### Example 1

The quercetin alkaline solution was prepared by dissolving 104.2 g quercetin aqueous slurry (5%) into 200 g cold water and 62 g cold sodium hydroxide (1N) at 20° C. Then, quercetin alkaline solution was slowly added to pre-treated gellan gum solution at pH 4 under high shear mixing. The dispersion pH was maintained 4 by adding citric acid during addition of quercetin alkaline solution. A homogeneous dispersion containing 0.5% quercetin was obtained. The concentrated quercetin dispersion was added to the beverage and suspended in the beverage.

Additional ingredients, including a high intensity natural sweetener were added to create a zero calorie orange-flavored isotonic beverage. The pH of the resultant beverage was about 3.5.

| | Amount |
|---|---|
| Ingredient | (% by wt.) |
| Water | 99.22% |
| Salt Blend | 0.176% |
| Quercetin | 0.1% |
| Gellan gum | 0.03% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 2

The quercetin alkaline solution was prepared by dissolving 104.2 g quercetin aqueous slurry (5%) into 200 g cold water and 62 g cold sodium hydroxide (1N) at 20° C. Then, quercetin alkaline solution was slowly added to pre-treated gellan gum solution at pH 5 under high shear mixing. The dispersion pH was maintained 4 by adding citric acid during addition of quercetin alkaline solution. A homogeneous dispersion containing 0.5% quercetin was obtained. The concentrated quercetin dispersion was added to the beverage and suspended in the beverage.

Additional ingredients, including a high intensity natural sweetener were added to create a zero calorie orange-flavored isotonic beverage. The pH of the resultant beverage was about 3.5.

| | Amount |
|---|---|
| Ingredient | (% by wt.) |
| Water | 99.22% |
| Salt Blend | 0.176% |
| Quercetin | 0.1% |
| Gellan gum | 0.03% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 3

The quercetin alkaline solution was prepared by dissolving 104.2 g quercetin aqueous slurry (5%) into 200 g cold water and 62 g cold sodium hydroxide (1N) at 20° C. Then, quercetin alkaline solution was slowly added to pre-treated gellan gum solution at pH 6 under high shear mixing. The dispersion pH was maintained 4 by adding citric acid during addition of quercetin alkaline solution. A homogeneous dispersion containing 0.5% quercetin was obtained. The concentrated quercetin dispersion was added to the beverage and suspended in the beverage.

Additional ingredients, including a high intensity natural sweetener were added to create a zero calorie orange-flavored isotonic beverage. The pH of the resultant beverage was about 3.5.

| | Amount |
|---|---|
| Ingredient | (% by wt.) |
| Water | 99.22% |
| Salt Blend | 0.176% |
| Quercetin | 0.1% |
| Gellan gum | 0.03% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 4

The quercetin alkaline solution was prepared by dissolving 104.2 g quercetin aqueous slurry (5%) into 200 g cold water and 62 g cold sodium hydroxide (1N) at 20° C. Then, quercetin alkaline solution was slowly added to pre-treated gellan gum solution at pH 7 under high mixing. The dispersion pH was maintained 4 by adding citric acid during addition of quercetin alkaline solution. A homogeneous dispersion containing 0.5% quercetin was obtained. The concentrated quercetin dispersion was added to the beverage and suspended in the beverage.

Additional ingredients, including a high intensity natural sweetener were added to create a zero calorie orange-flavored isotonic beverage. The pH of the resultant beverage was about 3.5.

| | Amount |
|---|---|
| Ingredient | (% by wt.) |
| Water | 99.22% |
| Salt Blend | 0.176% |
| Quercetin | 0.1% |
| Gellan gum | 0.03% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 5

The quercetin alkaline solution was prepared by dissolving 104.2 g quercetin aqueous slurry (5%) into 200 g cold water and 62 g cold sodium hydroxide (1N) at 20° C. Then, quercetin alkaline solution was slowly added to pre-treated gellan gum solution at pH 3 under high mixing. The dispersion pH was maintained 4 by adding citric acid during addition of quercetin alkaline solution. A homogeneous dispersion containing 0.5% quercetin was obtained. The concentrated quercetin dispersion was added to the beverage and suspended in the beverage.

Additional ingredients, including a high intensity natural sweetener were added to create a zero calorie orange-flavored isotonic beverage. The pH of the resultant beverage was about 3.5.

| | Amount |
|---|---|
| Ingredient | (% by wt.) |
| Water | 99.22% |
| Salt Blend | 0.176% |
| Quercetin | 0.1% |
| Gellan gum | 0.03% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 6

The resveratrol alkaline solution was prepared by dissolving 0.5 g resveratrol into 10 g cold sodium hydroxide (1N) at 20° C. Then, resveratrol alkaline solution was slowly added to pre-treated gellan gum solution at pH 4 under high mixing. The dispersion pH was maintained 4 by adding citric acid during addition of resveratrol alkaline solution. A homogeneous dispersion is obtained. The concentrated resveratrol dispersion was added to the beverage and suspended in the beverage.

Additional ingredients, including a high intensity natural sweetener were added to create a zero calorie orange-flavored isotonic beverage. The pH of the resultant beverage was about 3.5.

| | Amount |
|---|---|
| Ingredient | (% by wt.) |
| Water | 99.28% |
| Salt Blend | 0.176% |
| Resveratrol | 0.04% |
| Gellan gum | 0.03% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |

### Example 7

The rutin alkaline solution was prepared by dissolving 1 g rutin into 13 g cold water, 25.5 g sodium hydroxide (0.1N), and 1 g (1N) sodium hydroxide at 20° C. Then, rutin alkaline solution was slowly added to pre-treated gellan gum solution at pH 4 under high mixing. The dispersion pH was maintained 4 by adding citric acid during addition of rutin alkaline solution. A homogeneous dispersion is obtained. The concentrated rutin dispersion was added to the beverage and suspended in the beverage.

Additional ingredients, including a high intensity natural sweetener were added to create a zero calorie orange-flavored isotonic beverage. The pH of the resultant beverage was about 3.5

| | Amount |
|---|---|
| Ingredient | (% by wt.) |
| Water | 99.24% |
| Salt Blend | 0.176% |
| Rutin | 0.083% |
| Gellan gum | 0.03% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 8

The curcumin alkaline solution was prepared by dissolving 1 g curcumin into 10 g cold water, 25 g sodium hydroxide (0.1N), and 8 g (1N) sodium hydroxide at 20° C. Then, curcumin alkaline solution was slowly added to pre-treated gellan gum solution at pH 4 under high mixing. The dispersion pH was maintained 4 by adding citric acid during addition of curcumin alkaline solution. A homogeneous dispersion was obtained. The concentrated curcumin dispersion was added to the beverage and suspended in the beverage.

Additional ingredients, including a high intensity natural sweetener were added to create a zero calorie orange-flavored isotonic beverage. The pH of the resultant beverage was about 3.5

| | Amount |
|---|---|
| Ingredient | (% by wt.) |
| Water | 99.24% |
| Salt Blend | 0.176% |
| Curcumin | 0.083% |
| Gellan gum | 0.03% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 9

The naringenin alkaline solution was prepared by dissolving 1 g naringenin into 10 g cold water, 24 g sodium hydroxide (0.1N), and 5.7 g (1N) sodium hydroxide at 20° C. Then, naringenin alkaline solution was slowly added to pre-treated gellan gum solution at pH 4 under high mixing. The dispersion pH was maintained 4 by adding citric acid during addition of naringenin alkaline solution. A homogeneous dispersion is obtained. The concentrated naringenin dispersion was added to the beverage and suspended in the beverage.

Additional ingredients, including a high intensity natural sweetener were added to create a zero calorie orange-flavored isotonic beverage. The pH of the resultant beverage was about 3.5.

| | Amount |
|---|---|
| Ingredient | (% by wt.) |
| Water | 99.24% |
| Salt Blend | 0.176% |
| Naringenin | 0.083% |
| Gellan gum | 0.03% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

### Example 10

The hesperedin alkaline solution was prepared by dissolving 1 g hesperedin into 15 g cold water, 26 g sodium hydroxide (0.1N), and 3.5 g (1N) sodium hydroxide at 20° C. Then, hesperedin alkaline solution was slowly added to pre-treated gellan gum solution at pH 4 under high mixing. The dispersion pH was maintained 4 by adding citric acid during addition of hesperedin alkaline solution. A homogeneous dispersion was obtained. The concentrated hesperedin dispersion was added to the beverage and suspended in the beverage.

Additional ingredients, including a high intensity natural sweetener were added to create a zero calorie orange-flavored isotonic beverage. The pH of the resultant beverage was about 3.5.

| | Amount |
|---|---|
| Ingredient | (% by wt.) |
| Water | 99.24% |
| Salt Blend | 0.176% |
| Hesperedin | 0.083% |
| Gellan gum | 0.03% |
| Citric Acid | 0.180% |
| Mango Flavor | 0.100% |
| Yellow #6 Color 10% solution | 0.060% |
| Liq. Sucralose (25%) | 0.021% |
| Ace K | 0.003% |
| Vitamin C (Ascorbic Acid) | 0.105% |
| Vitamin B3 (Niacinamide) | 0.004% |
| Total | 100.000% |

The Table below demonstrates the effect of gellan gum hydration pH on Quercetin dispersion stability

| | Gellan Gum | Q Dispersion | Q Dispersion |
|---|---|---|---|
| | Hydration | Stability at | Stability |
| Example | pH | 90° F. | at 70° F. |
| 5 | 3 | 15 days, settling | at least 2 |
| | | and phase | months |
| | | separation | |
| 1 | 4 | at least 30 days | at least 2 |
| | | with no settling | months |
| | | and phase | |
| | | separation | |
| 2 | 5 | 10 days, settling | at least 2 |
| | | and phase | months |
| | | separation | |
| 3 | 6 | 6 days, settling | at least 1 |
| | | and phase | month |
| | | separation | |
| 4 | 7 | 3 days, settling | at least 10 |
| | | and phase | days |
| | | separation | |

This invention may be embodied in other specific forms without departing from the spirit or essential characteristics thereof. The foregoing embodiments, therefore, are to be considered in all respects illustrative rather than limiting the invention described herein. The scope of the invention is thus indicated by the appended claims, rather than by the foregoing description, and all changes that come within the meaning and range of equivalency of the claims are intended to be embraced therein.

We claim:

1. A method for preparing a stable dispersion of microparticulated water insoluble bioactive polyphenol comprising the steps of:
a. dissolving at least one water insoluble bioactive polyphenol in an alkaline solution at a temperature of less than 30° C., wherein the alkaline solution has a pH of 9 to 12, to form an alkaline bioactive polyphenol solution;
b. forming an aqueous solution containing a dispersion stabilizer, wherein the aqueous solution has a pH of 3 to 5 and a temperature below 50° C.; and
c. introducing the alkaline bioactive polyphenol solution into the stabilizer aqueous solution containing the dispersion stabilizer while maintaining the aqueous dispersion at the pH of 3 to 5 and the temperature below 50° C., thereby forming a dispersion of water insoluble bioactive polyphenol particles in the aqueous solution, wherein the particles are microparticulated;
wherein the dispersion stabilizer is present in an amount sufficient to stabilize the dispersion of water insoluble bioactive polyphenol particles.

2. The method of claim 1 wherein the temperature of the alkaline solution is 2 to 20° C.

3. The method of claim 1 wherein the temperature of the alkaline solution is 5 to 15° C.

4. The method of claim 1 wherein the temperature of the alkaline solution is 5 to 10° C.

5. The method of claim 1 wherein the alkaline solution has a pH of 9.5 to 11.5.

6. The method of claim 1 wherein the alkaline solution comprises sodium hydroxide, potassium hydroxide, ammonium hydroxide, sodium carbonate, sodium hydrogenate carbonate, sodium bicarbonate, sodium hydrogen orthophosphate, potassium chloride, citric acid, mixtures thereof.

7. The method of claim 1 wherein the alkaline solution comprises one of the following combinations: sodium hydroxide and potassium hydroxide; sodium carbonate and sodium hydrogen carbonate; sodium bicarbonate and sodium hydroxide; sodium hydrogen orthophosphate and sodium hydroxide; potassium chloride and sodium hydroxide; and citric acid and sodium hydroxide.

8. The method of claim 1 wherein the at least one water insoluble bioactive polyphenol is at least one flavonoid.

9. The method of claim 8 wherein the at least one flavonoid is selected from the group consisting of flavonones, flavones, dihydroflavonols, flavonols, flavandiols, leucoanthocyanidins, flavonol glycosodes, flavonone glycosides, isoflavonoids, and neoflavonoids.

10. The method of claim 1 wherein the at least one water insoluble bioactive polyphenol is selected from the group consisting of quercetin, eriocitrin, neoeriocitrin, narirutin, naringin, hesperidin, hesperetin, neohesperidin, neoponcirin, poncirin, rutin, isorhoifolin, rhoifolin, diosmin, neodiosmin, sinensetin, nobiletin, tangeritin, catechin, catechin gallate, epigallocatechin, epigallocatechin gallate, anthocyanin, heptamethoxyflavone, curcumin, resveratrol, naringenin, tetramethoxyflavone, kaempferol, rhoifolin, and oolong tea polymerized polyphenol.

11. The method of claim 1 wherein the at least one water insoluble bioactive polyphenol is selected from the group consisting of curcumin, rutin, resveratrol, naringenin, hesperedin, and tetramethoxyflavone.

12. The method of claim 1 wherein the at least one water insoluble bioactive polyphenol is quercetin.

13. The method of claim 1 wherein the at least one dispersion stabilizer is an anionic or cationic biopolymer or a modified polysaccharide selected from the group consisting of gellan gum, pectin, guar gum, xanthan gum, acacia gum, locust gum, agar, starch, ghatti gum, carrageenan, alginate, cellulose, protein, hydrolyzed protein, modified starch, carboxyl methyl cellulose, or mixtures thereof.

14. The method of claim 13 wherein the at least one dispersion stabilizer is a gellan gum, pectin, gum arabic, ghatti gum, carrageenan, alginate, CMC, whey protein isolate, or mixtures thereof.

15. The method of claim 1 wherein the aqueous solution containing at least one dispersion stabilizer is a beverage.

16. The method of claim 15 wherein the dispersion stabilizer is present in the beverage in a concentration of about 0.001-5.0 wt. % based on total weight of the beverage.

17. The method of claim 15 wherein the water insoluble bioactive polyphenol particles comprise 0.01 to 1 wt % of the final beverage composition.

18. The method of claim 15 wherein the beverage has a pH of less than 6.

19. A method for dispersing polyphenol particles in a beverage comprising a) dissolving at least one water-insoluble polyphenol in an alkaline solution at a temperature below 30° C.; and b) adding the alkaline solution to an aqueous beverage composition containing a dispersion stabilizer having a pH of 3 to 5 and a temperature of less than 50° C. thereby forming a microparticulated polyphenol dispersion; wherein the dispersion stabilizer is present in an amount sufficient to stabilize the dispersion of microparticulated polyphenol in the beverage.

20. The method of claim 19 wherein the polyphenol is quercetin.

21. The method of claim 19 wherein the at least one dispersion stabilizer is an anionic or cationic biopolymer.

22. The method of claim 21 wherein the at least one dispersion stabilizer is a gellan gum, gum arabic, pectin, carrageenan, ghatti gum, alginate, CMC, whey protein isolate, or mixtures thereof.

23. A composition for a comestible comprising dispersed microparticulated polyphenol and at least one dispersion stabilizer in an aqueous solution, wherein about 90% of the polyphenol has a particle size below about 50 microns and the at least one dispersion stabilizer is present in an amount sufficient to stabilize the dispersion of microparticulated polyphenol in the aqueous solution.

24. The composition for a comestible of claim 23 wherein the polyphenol is quercetin.
